(19) United States
(12) Patent Application Publication
(10) Pub. No.: US 2012/0071702 A1
(43) Pub. Date: Mar. 22, 2012

(54) Chemical Reactor System and Method Using Regenerative Turbine Pump to Produce Fuel Gas

(76) Inventor: James Charles BUTLER, Cherry Hill, NJ (US)

(21) Appl. No.: 13/240,836

(22) Filed: Sep. 22, 2011

Related U.S. Application Data
(60) Provisional application No. 61/385,423, filed on Sep. 22, 2010.
(60) Provisional application No. 61/385,392, filed on Sep. 22, 2010.

(51) Int. Cl. C07C 1/20; B01J 10/00

(52) U.S. Cl. 585/733; 422/129; 422/187; 422/105; 422/112; 422/162

(57) ABSTRACT

A pump system and controller for a chemical reactor for converting water and carbon dioxide into a fuel gas is provided. Carbon dioxide gas bubbles are created and introduced into pumped water and delivered to a regenerative turbine pump where bubbles are collapsed to produce an ionized gas and ionized liquid mixture containing hydrogen, hydroxyl radicals and hydroxide, which subsequently react with the carbon dioxide gas present in the bubble, reducing it to carbon monoxide, with further reactions yielding methane.

### CROSS-REFERENCE TO RELATED APPLICATION

This application claims the benefit of the priority of U.S. Provisional Application Ser. No. 61/385,423, filed Sep. 22, 2010, and United States Provisional Application Ser. No. 61/385,392, filed Sep. 22, 2010, the entire disclosures of which are expressly incorporated herein by reference.

### FIELD OF THE INVENTION

The present invention relates to chemical reactors and pump systems and more specifically to a chemical reactor having a regenerative turbine pump for producing fuel gas.

### BACKGROUND OF THE INVENTION

Endothermic synthesis reactions convert water and carbon dioxide to a fuel gas mixture containing carbon monoxide, methane and hydrogen. Such reactions include the reverse water gas shift, the Sabatier reaction and the Fischer-Tropsch process.

Cavitation caused by ultrasonic and hydrodynamic equipment and techniques has been used for catalysis of many known and well defined endothermic reaction traditionally implemented with high temperature/high pressure processes for many years. The advantage of cavitation as a mechanism of chemical catalysis is the effect of high pressure and temperatures achieved in the domain of effect near the collapsed bubble, the rate of heating being sufficient to start and sustain reactions in aqueous and other solutions.

Reduction and oxidation synthesis has been implemented using high pressure/high temperature reactors for decades. More recently, in the field of ultrasonic sonochemistry, some of these reactions have been duplicated in experimental configurations; the results reported in the literature suggest that sonochemical reduction catalysis, specifically hydrolysis resulting in protonation of carbon dioxide in aqueous solutions, reducing it to carbon monoxide, can be catalyzed under specific conditions using ultrasonic cavitation. As this mechanism appears effective for reduction catalysis, it follows that further protonation using cavitational techniques or methods analogous to cavitational chemical catalysis result in the production of CH₄ and other alkanes.

Implementation of hydrodynamic cavitation and sonochemistry for commercial or industrial scale chemical synthesis has arguably been curtailed by the nature of the mechanism used to catalyze reactions for these purposes—cavitation. While single bubble collapse as achieved in single bubble sonoluminescence has been demonstrated as a stable, controlled bubble collapse technique using the methods of ultrasonic cavitation, cavitation as applied in industrial processes almost invariably use multi-bubble cavitation as the catalytic mechanism. This technique, when used at high power levels on materials containing carbon dioxide, such as fossil fuel combustion waste gas streams, surface or pitted waste waters, process effluents or seawater with significant quantities of dissolved carbon dioxide and carbonic acid require continuous modification of process conditions to sustain economically feasible throughput rates. These types of changes to cavitation bases mechanism are difficult as both ultrasonic and hydrodynamic cavitation require limited and specific conditions to achieve the high temperature and pressure collapse characteristics required to sustain productive rates of specific reaction catalysis.

Ultrasonic sonochemical processes are dependent on specific frequencies, or specific ultrasonic wave generation surface or horn travel distances or both. Once these variables are optimized in process, changes to reagent composition, ambient pressures, temperature, dissolved gasses or other solvent or solute properties often immediately destabilize the bubble cloud formation and collapse, significantly halting or slowing reaction catalysis, rendering the continued application of the technique without process variable changes uneconomic. Adjustments to frequency and amplitude of the ultrasonic field directly affect the properties of the created and collapsing bubble, changing the resonant frequency. The changes to frequency or amplitude, ambient pressure, temperature, the amount of carbon dioxide or carbonic acid in solution, or solution pH, may result in conditions that, while producing cavitation, produce bubbles with properties of size, resonant frequency or collapse rate that are not optimal for the catalyzing economic reduction of carbon dioxide to alkane fuel gasses. This limitation is due to the dependence of bubble properties on ultrasonic field frequency and amplitude. The bubble properties can be modulated through advanced process control of these process parameters, but only certain frequency and amplitude combinations are effective. As a consequence, reactive closed loop control of chemical synthesis through reduction based on this mechanism is limited in the scope of variation, detrimentally affecting the economics of processes using this mechanism of reaction catalysis.

There are many hydrodynamic cavitation implementation techniques using a variety of methods to manipulate the formation and collapse of bubbles through pressure and flow control. In addition, custom fittings and shaped orifices and other fixture based techniques for laminar and other unusual flow patterns, with and without closed loop control, have been in used to produce cavitation and catalyze compositional degradations and synthesis. Hydrodynamic cavitational techniques also suffer from restrictions due to optimal condition requirements similar to ultrasonic cavitation, where variation in composition, viscosities, dissolved or suspended solids content, dissolved gasses and other properties of the solvent or solute detrimentally affect the properties and formation of the clouds of bubbles and their subsequent collapse rates, shapes and characteristics.

Both ultrasonic and hydrodynamic cavitation also result in cavitational damage to process circuit elements as conditions for controlled cavitation often result in insipient or other undesirable cavitational effects at or on surfaces or system components, resulting in component wear, significantly affecting process economics.

Carbon dioxide and water are often, by design, the final products of many synthesis processes. Many industrial activities and process circuits yield carbon dioxide as a product, such as fossil fuel combustion for energy and heating, iron and steel production, cement manufacturing, natural gas systems, municipal solid waste combustion and many others. Recent measurements indicate U.S. production of carbon dioxide in excess of five thousand megatons per year. See U.S. EPA website, Climate Change—Greenhouse Gas Emissions page, http://www.epa.gov/climatechange/emissions/co2_human.html

Carbon dioxide released into the atmosphere undergoes a continuous process of equilibrium exchanges between the gaseous state as a component gas of the atmosphere, dissolved carbon dioxide in surface waters and carbonic acid in surface waters. The solubility of carbon dioxide is higher in salt water than fresh water, thus a significant quantity of carbon dioxide released into the environment arguably currently resides in the oceans and other surface salt water. See the National Oceanic and Atmospheric Administration PMEL website, Carbon Program, Ocean Carbon Update page, http://www.pmel.noaa.gov/co2/story/Ocean+Carbon+Uptake and National Oceanic and Atmospheric Administration PMEL website, Carbon Program, Ocean Acidification page, http://www.pmel.noaa.gov/co2/story/Ocean+Acidification.

As a consequence of the production of significant amounts of carbon dioxide over the years, and the dissolution of carbon dioxide into surface waters over time, a method that can be used to harvest carbon dioxide from emissions sources, the atmosphere directly and surface waters and subsequently convert the harvested carbon dioxide to recoverable fuel gasses or liquids would provide a new and renewable energy supply. Current solutions and techniques for carbon dioxide removal focus almost entirely on atmospheric carbon dioxide and contemplate methods to sequester or store carbon dioxide in perpetuity at great expense without the benefit of recycling or reuse. The economics of these approaches render them likely unfeasible, especially considering that the scale of carbon dioxide processing contemplated is megatonnage.

### SUMMARY OF THE INVENTION

The invention provides a solution to the aforementioned limitations of both hydrodynamic and ultrasonic cavitation and a means to effectively and economically recover and recycle carbon dioxide using an approach scalable to megaton gas processing and suitable for application to gaseous, liquid and solid feedstocks containing carbon dioxide. Rather than causing bubble collapse through cavitation, the invention provides methods and configurations to produce bubbles in a controlled way directly and then, also in a directly controlled way, collapse those bubbles at a specific rate to a specific size. In addition, the bubbles collapse while entrained in fluid flow, preventing undesirable bubble collapse upon process component surfaces. As the formation and collapse of the bubbles in the invention occurs independent of optimal flow, pressure, temperature, viscosity, and other solvent and solute properties, and the bubble sizes and collapse rates are directly controlled and not a function of effective ultrasonic amplitudes or frequencies, or fixed pressure or flow established in specialized fittings, this mechanism of bubble collapse can be applied to catalyze reactions economically across a broader range of carbon dioxide containing feedstock variability. The invention also provides methods and systems to accept a gas stream containing carbon dioxide and dissolve it in an aqueous solution to permit processing and methods and systems to process and reduce carbon dioxide and carbonic acid in aqueous solutions, such as seawater, permitting the application for carbon dioxide conversion to fuel gas at both point sources of gas emission, such as power plants, and from environmental resources, such as seawater, using the same apparatus and methods of chemical catalysis and synthesis.

A pump system and controller for a chemical reactor for converting water and carbon dioxide into a fuel gas are disclosed. One aspect of the invention provides methods and apparatus to produce carbon dioxide gas combined with water vapor bubbles and at a rapid rate, introducing them in a controlled fashion into pumped water in a regenerative turbine pump and then collapsing the bubbles produced to convert the internal water vapor of the bubbles into an ionized gas and ionized liquid mixture containing hydrogen, hydroxyl radicals and hydroxide, which subsequently react with the carbon dioxide gas present in the bubble, reducing it to carbon monoxide, with further reactions yielding methane. The carbon dioxide, carbon monoxide or methane is reacted with hydrogen to produce alkane gasses, such as propane or butane.

Another aspect of the invention provides methods and apparatus that use the properties of collapsing bubbles, formed of carbon dioxide gas and water vapor, in water, as a catalyst for reactions between the water vapor and liquid, hydrogen and carbon dioxide in the gas and liquid phases of the bubble, or as a catalyst for reactions between liquid water, water vapor, carbon, hydrogen, carbon dioxide and carbon monoxide gasses contained in the bubble and the water, hydrogen, hydroxyl radicals and hydroxide in the pumped bubble containing water media.

Another aspect of the invention provides methods and apparatus to form carbon dioxide gas and water vapor containing bubbles in a pumped water media and to collapse them in isolation from each other using a controlled hydrodynamically generated pressure pulse of sufficient magnitude, both in rate of pressure increase and ultimate maximum pressure that the bubble vibrates, for example at its eigenfrequency, during collapse for a sufficient interval of time to permit the formation of a plasma hot spot within the gas and liquid phases of the collapsing bubble.

Yet another aspect of the invention provides a device that permits concentrations of carbon dioxide and water vapor of the bubbles formed in a pumped media at a pump system inlet to be both directly and indirectly controlled, at the time of formation and during evolution through the various possible bubble sizes through to collapse, as the bubbles pass from the pump inlet's low pressure zone through the pump at increasing pressure and out the discharge at a particular controlled maximum pressure.

Still another aspect of the invention provides a pump system based on a regenerative turbine pump with components arranged to allow controlled bubble production and introduction into the pump inlet and subsequent collapse of the bubbles entrained in the helical flow of the pump within the individual bucket chambers formed by the regenerative turbine pump impeller, wherein the bubbles are collapsed singly without the interfering effects of the collapse of adjacent bubbles.

Another aspect of the invention provides methods and apparatus for electric motor driven pump speed and pressure control. The pump control system dynamically calculates optimal pump speed and pump system pressures for one of the alternate apparatus configurations or applications, to start and sustain the formation in the pumped media of a specific number of gas and vapor bubbles of a particular size and then subsequently collapse the same bubbles at a particular rate to a specific ultimate final bubble size. The pump control system incorporates a controller that provides a speed setpoint signal to the pump motor drive and pressure setpoint signals used to operate pressure regulating valves controlling pump inlet, casing and discharge pressures.

The controller can be used as an analytical tool to determine the optimal operational parameter values required for producing bubbles of a certain size and collapsing them at a specific rate to plasma hot spots or as required by a particular application of the invention. In this way, an application protocol describing the operational conditions and process variable selections most likely to produce a desired result with the apparatus can be developed using the apparatus and controller as reporting those setpoint combinations yielding desirable or best fit operational characteristics using controller residing result evaluation algorithms.

The following description, with attached diagrams, provides details of the important aspects of the invention. Note, however, that the invention has other useful and novel aspects apart from those discussed. These additional aspects and advantages of the invention will become apparent when considering together the following detailed description and drawings.

### BRIEF DESCRIPTION OF THE DRAWINGS

FIG. 1 shows the interconnection between FIGS. 1A and 1B;

FIGS. 1A and 1B show a piping and instrumentation diagram shown over two pages depicting an exemplary implementation of the apparatus of the present invention including a pump system, subsystems, components and apparatus controller used to inject carbon dioxide gas bubbles into water and subsequently collapse them in a regenerative turbine pump to form a fuel gas which is separated from the pumped fluid;

FIGS. 2A-2C are cross-sectional views of the regenerative turbine pump's casing and impeller blade channel;

FIG. 3 is a flowchart showing examples of the controller's logic, including alternate operational sequences as required by the various functional configurations of the apparatus of the present invention, in accordance with several aspects of the invention;

FIG. 4 is a flowchart showing processing steps carried out by a carbon dioxide reduction logic of the present invention;

FIG. 5 is a flowchart showing processing steps carried out by a bubble generation logic of the present invention; and

FIG. 6 is a flowchart showing processing steps carried out by a bubble collapse logic of the present invention.

### DETAILED DESCRIPTION OF THE INVENTION

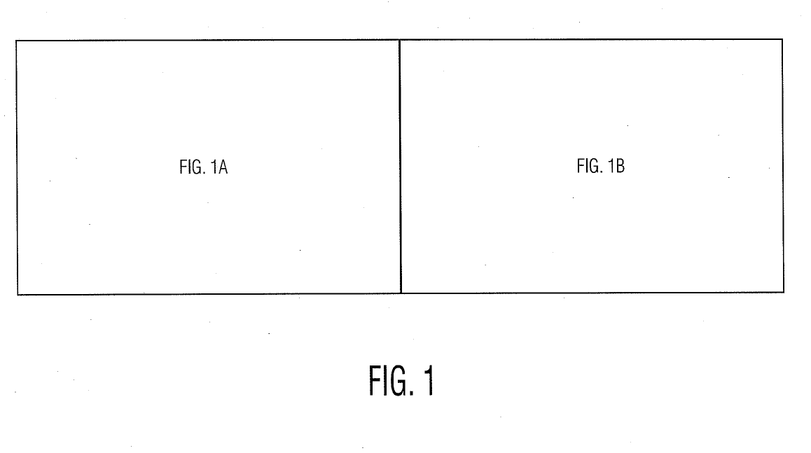

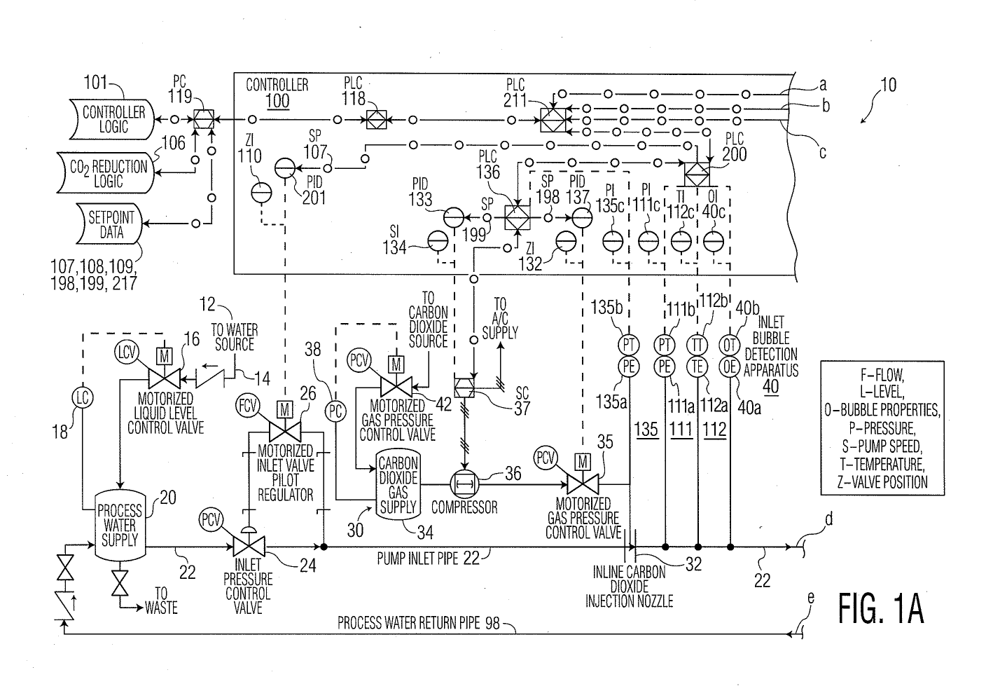

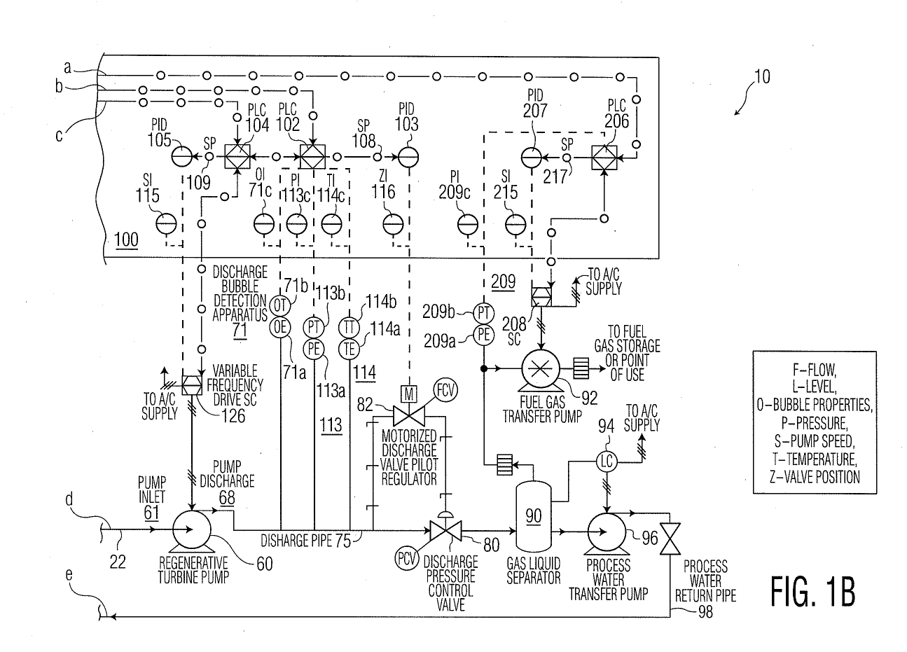

The apparatus of the present invention includes several independent subsystems that together provide for the production and injection of carbon dioxide gas into a water stream to form bubbles, and collapse of the bubbles at a controlled rate to plasma, reducing the carbon dioxide to fuel gas. The apparatus includes a regenerative turbine pump to effectively entrain and collapse bubbles. FIGS. 1A and 1B depict an exemplary piping and instrumentation layout of the apparatus 10, including subsystems, shown separated and shown over two pages.

A controlled linear stream of carbon dioxide bubbles, which could be approximately 0.5 micron to 1 mm diameter in size, is injected into a pumped water media at the inlet, but outside of the casing, of a regenerative turbine pump. The carbon dioxide bubbles are then collapsed, to approximately 0.1 um to 0.2 um, in less than approximately 4 milliseconds with a pressure rise of approximately 1,000 psig, for example, while entrained in the helical pumped media flow within the regenerative turbine pump impeller's blade channel so that a plasma hot spot is formed at the center of each collapsed bubble. In this way carbon dioxide gas is brought into close proximity to hydrogen released from water at the high pressure reached at the core of the collapsed carbon dioxide bubble (approximately 7,200 psig, for example) and the commensurate collapsed bubble domain's high temperature (greater than approximately 2,000° C., for example), resulting in carbon dioxide reduction to carbon monoxide and methane.

Referring to FIGS. 1A and 1B, an exemplary apparatus is generally indicated at 10. Proceeding from the apparatus inlet downstream through to the apparatus discharge, the liquid flow into the apparatus begins with an inlet pipe 14 connected between water source 12 and process water supply tank 20. Delivery pressure at the inlet pipe 14 centerline is preferably equal to or greater than the net positive suction head required (NPSHr) by the regenerative turbine pump 60 at its expected maximum flow during carbon dioxide reduction and is also preferably greater than the expected maximum pressure required by the bubble generation apparatus 30, approximately 3 to 5 psig, for example.

When the system is initially started, the process water supply tank 20 is filled from the water source to an initial level as controlled by the process water supply level controller 18. After initial fill and during system operation process water is recirculated from the gas liquid separator 90 and returned to the process water supply tank 20. Insufficient suction head at the inlet pipe 14 can be overcome through the addition of a water booster pump (not shown) between the process water supply tank 20 and the inlet pressure control valve 24. During operation, process water is converted to hydrogen and oxygen and reacted. Make-up water is added to the process water supply tank 20 as determined by the level controller 18 through motorized liquid level control valve 16 to restore water consumed by the carbon dioxide conversion reaction.

When the regenerative turbine pump 60 is operating, the process water flows from the process water supply tank 20, through the pump inlet pipe 22, then through an inlet pressure control valve 24 with a motorized inlet valve pilot regulator 26 for controlling flow. The controller 100 actuates the motorized inlet valve pilot regulator 26 as directed by controller logic 101 and carbon dioxide reduction logic 106 in response to conditions in the pumped water at the pump inlet 61, such as pressure and temperature measured by pump inlet pressure 111 and temperature 112 sensors. Pump inlet pressure sensor 111 may include pump pressure element 111a, pump pressure transmitter 111b and pump pressure indicator 111c. Temperature sensor 112 may include temperature element 112a, temperature transmitter 112b and temperature indicator 112c. The sensors comprise a detector element to measure and a transmitter to send the value. While the sensors are shown as discrete devices, the sensors could be a single device. Values sent from transmitters are shown to users on indicators, which can be local or remote gauges or computer graphical monitors. An example of a suitable commercially available pressure sensor/transmitter is Ashcroft Xmitr, 0-100 psi, 3″. The primary control algorithms, operation and detection sequencing instructions and setpoints or setpoint algorithms, are stored in the controller, which can be a PC, Panel-PC, PLC (programmable logic controller) or some other specific purpose programmable HMI (human-machine interface) device or controller. An example of a suitable commercially available PLC is Allen-Bradley Micrologix 1400 Model 1766-L32BWAA. An example of a suitable commercially available PLC software with PID algorithms is RSLogix 500 Professional. An example of a suitable commercially available PC is HP Compaq dx2450.

The controller 100 calculates and adjusts the process water pressure setpoint downstream of the inlet pressure control valve 24 by adjusting the inlet pressure control valve 24 using the motorized inlet valve pilot regulator 26. These adjustments can be made using the pump inlet pipe 22 process water condition information gathered by the pump inlet pipe sensors 40, 111 and 112, regulating pressure as required by the inlet bubble generation, apparatus 30 and enabling sustained generation carbon dioxide bubbles having a size of approximately 0.5 micron to 1 mm in diameter, for example. Inlet bubble detector sensor 40 may include sensor element 40a, transmitter 40b and indicator 40c. The controller 100 can detect pump inlet pipe 22 pressure, temperature and generated bubble properties, and regulate apparatus inlet pressure, and calculate a new position setpoint for the motorized inlet valve pilot regulator 26, which in turn adjusts the position of the inlet pressure control valve 24, regulating the pump inlet pipe 22 pressure at the bubble generation apparatus 30. In this way, changes in liquid supply temperature, pressure or other parameter values that occur during the operation of the apparatus, such as a level drop in a water supply 20 tank, pump inlet pipe 22 turbulence, performance change in an upstream connected process, change in liquid supply pressure, or a change in the flow rate requirements of the downstream subsystems can be detected and compensated for using closed-loop control, maintaining pressure and flow as required by the bubble generation apparatus 30.

Continuing with FIGS. 1A and 1B, the process water flows from the inlet pressure control valve 24 through the inlet bubble generation apparatus 30, where carbon dioxide gas is injected into the inlet pipe forming bubbles in the process water. Any suitable bubble production method using suitable apparatus and methods can be employed. It is expected that a useful configuration of the inlet bubble generation apparatus 30 will produce a steady, linear bubble stream in the range of approximately 9,000 to 10,000 bubbles per second, for example, with stable bubble diameters in the range of approximately 0.5 microns to 1 mm, for example. The aforementioned ranges can vary for different operating conditions. Also, it is desirable for the bubble generation apparatus 30 to produce a linear stream of bubbles as opposed to uncontrolled clouds of bubbles produced by some types of bubblers and elements of cavitation reactors, even if the number and size of bubbles in the cloud are controlled. The following configuration of the bubble generation apparatus 30, while not representative of all possible alternatives as might be required for carbon dioxide reduction under all operating conditions, provides for bubble production in sufficient number, size, and placement.

The bubble generation apparatus 30 uses an injection nozzle 32, which is sized and shaped to produce bubbles based on flow rate and throughput of the system, installed in the pump inlet pipe 22 line, pointed downstream and axially centered in the pump inlet pipe 22 before the inlet bubble detection apparatus 40 or the regenerative turbine pump 60, optionally followed by a vortex fitting (not shown) such as, for example, a standard NIBCO vortex insertion feeder manufactured by NIBCO, Inc., from Elkhart, Ind., or a Steinen Tan-Jet nozzle, manufactured by Wm. Steinen Mfg. Co., based in Parsippany, N.J., to assist bubble entrainment. In this arrangement the carbon dioxide gas supply 34 is connected to a compressor 36 which discharges the gas into a pressure regulated line. Inline gas injection nozzle pressure and compressor speed control programmable logic controller (PLC) 136 reads the ranged analog or digital injector pressure signal from the inline gas injection nozzle pressure 135 which may include pressure element 135a, pressure transmitter 135b and pressure indicator 135c. Gas injection nozzle PLC 136 continuously retrieves the current inline gas injection nozzle pressure setpoint 198 and recalculates a new pressure setpoint, transmitting a ranged analog or digital signal to the motorized gas pressure control valve 35 that regulates the injection nozzle pressure. If the inline gas injection nozzle pressure setpoint 198 cannot be achieved by the carbon dioxide gas supply 34 pressure, as regulated by the gas supply pressure controller 38, which controls motorized gas pressure control valve 42, then PLC 136 starts the compressor 36, and using the gas injection nozzle pressure at sensor 135 as the process variable, upwardly adjusts the inline gas injection nozzle compressor speed setpoint 199 until the inline gas injection nozzle pressure setpoint 198 is fractionally exceeded. Fine downward gas pressure adjustment is accomplished by changing the pressure setpoint transmitted to the motorized gas pressure control valve 35. Once PLC 136 calculates a pressure setpoint, it is transmitted to inline gas injection nozzle pressure control proportional-integral-derivative controller (PID) 137, with injection nozzle pressure as the process variable. PLC 136 relays the current speed of the motor (not shown) of compressor 36 from the compressor speed controller 37 to inline gas injection nozzle compressor speed control PID 133 along with the current compressor speed setpoint 199. This enables bubble generation across a wide range of operating pressures, and direct control of bubble content, size and number by varying the inline gas injection nozzle pressure setpoint 198 or the inline gas injection nozzle 32 shape or size, in addition to pressure of the liquid in the pump inlet pipe 22 regulated by the inlet pressure control valve 24.

Other bubble generator apparatus may be used instead of directly injecting bubbles into the fluid, as disclosed in commonly owned copending U.S. patent application Ser. No. ______, titled “Chemical Reactor System and Methods to Create Plasma Hot Spots in a Pumped Media,” and filed contemporaneously herewith. The disclosure of this application is expressly incorporated herein by reference. For example, gas bubbles could be aspirated through an eductor comprising a venturi having a side port for gas admittance. Pressure control before and after the venturi allows for the production of bubbles at a controlled rate. Alternatively, a venturi could be used to generate bubbles.

Next, the process water containing entrained carbon dioxide bubbles passes through the inlet bubble detection apparatus 40. Under fixed or low flow operating conditions, or with feedstock that is invariant in composition, it may be sufficient to monitor the bubble generation apparatus 30 operating conditions as reported by inlet pressure 111 and temperature 112 sensors without need for a bubble detection apparatus where the process output is suitable. Where such correlations are inadequate, or where bubble production parameter values must be more precisely controlled, additional analog or digital detection of the size and number of bubbles actually created may be required. In these applications, the controller 100 uses controller logic 101 that receives the analog or digital bubble production data signal output from the inlet bubble detection apparatus 40, and in conjunction with other data from inlet sensors 111 and 112 data, calculates new operational parameter values, such as the pressure of the inlet bubble generating inline carbon dioxide injection nozzle 32, establishing closed-loop bubble production control. The controller logic 101 could be programmed using any suitable high or low-level computer programming language, and could be embodied as computer-executable instructions stored in a computer-readable medium, such as flash memory or other type of non-volatile memory. The inlet bubble detection apparatus 40 can be any one of a number of devices that are designed to produce a ranged analog or digital signal that corresponds to the number and sizes of bubbles present in a particular location in a two phase liquid media, such as an interferometric laser imaging sizer, a broadband sound velocimeter, Doppler Sonography, or another acoustic technique for bubble sizing. Properties of the pumped media such as composition, opacity, inclusions, temperature, as well as the size and number of bubbles to be produced and the intended function of the invention will guide the selection of the appropriate inlet bubble detection apparatus 40 to be used in conjunction with a particular application.

Next, the process water containing the entrained carbon dioxide bubbles passes from the inlet bubble detection apparatus 40 and enters the regenerative turbine pump 60. The turbine pump design with pump inlet, discharge, casing, channel layout, and connecting piping arrangements and low internal clearances enables the regenerative turbine pump to entrain carbon dioxide gas bubbles in a helical flow of the pumped water within the pump casing's channel, preventing the bubbles from adhering to or forming on the internal surfaces of the pump or where the pumped bubble containing liquid media flows.

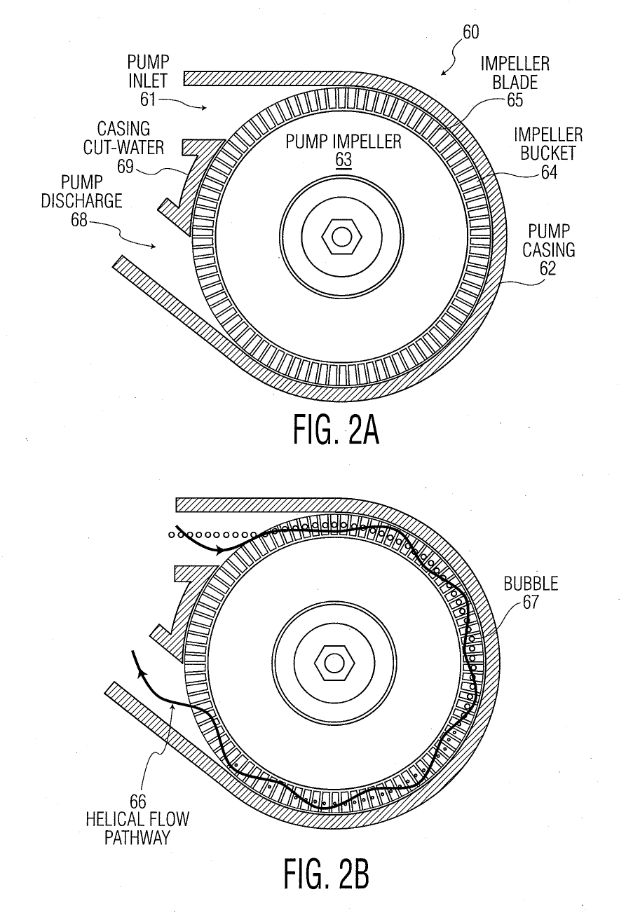

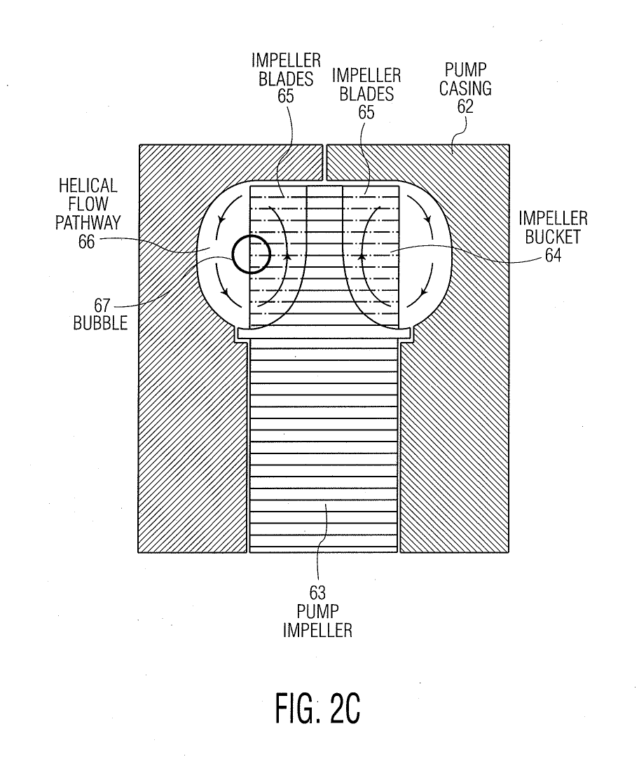

Referring now to FIGS. 2A-2C, the bubbles and process water pass through the pump inlet 61, which preferably has a smooth, straight-walled bore with minimal change in internal diameter across the pumped media flow path and which preferably intersects the casing tangentially, so that turbulence within the pump inlet, and consequently disruption to bubble size and location in the flow path, is minimized. The pumped media then passes into the regenerative turbine pump's casing 62 where the process water is forced, by containment within the impeller's buckets 64 formed by impeller blade 65 within the pump casing's annular space, to flow in a helical pathway 66. This occurs as the pumped liquid moves with the pump impeller 63 through the pump casing 62. The process water rotates about the axis of the direction of flow, causing an entrained bubble 67 to remain at the center of the flow path, preventing its adhesion to the pump casing's 62 internal surfaces and restraining it to a single impeller bucket 64.

The rotational speed setpoint of the pump impeller 63 required to establish stable and complete carbon dioxide conversion is maintained and adjusted as required by the controller 100 (see FIGS. 1A and 1B) during apparatus operation. The rotational speed is set so that the number of impeller buckets 64 that pass by the pump inlet 61 per second closely matches the number of flow entrained bubbles that pass through the pump inlet 61 per second. In addition, the pump speed setpoint and the regulated pump inlet and discharge pressure setpoints are those values that cause the process water flow rate to be such that the carbon dioxide bubbles are moved singularly into the impeller buckets 64, minimizing or eliminating the incidence of impeller buckets 64 that contain multiple bubbles or none during operation. Finally, the pump impeller 63 rotational speed and pump discharge 68 pressure are adjusted and maintained during operation that the bubbles individually entrained in the process water flow, and contained within individual impeller buckets 64, are collapsed as the surrounding process water flows from the pump inlet 61 to the pump discharge 68 at cut-water 69 and the pressure within the bubble containing impeller buckets 64 increases from the pump inlet 61 pressure to the final regulated pump discharge 68 pressure. In this way the initial and final bubble sizes, rate of bubble collapse, and final collapsed bubble core temperature and pressure, and consequently the rate of carbon dioxide reduction, is directly and mechanically controlled. The actual rate of bubble collapse is determined by the difference between the initial and final bubble size and the rate of pressure rise within the pump casing 62. Direct, automatic, mechanical control of these parameter values allows the apparatus function to be modulated, both initially and dynamically during operation, for optimal carbon dioxide reduction.

An example of a regenerative turbine pump for use in the present apparatus is the Regenerative Turbine Chemical Pump made by Roth Pump Company, Rock Island, Ill. Roth regenerative turbine chemical duty pumps provide continuous, high pressure pumping of non-lubricating and corrosive liquids. These regenerative turbine pumps are provided with one piece, machined self-centering impellers for operation with a wide variety of chemicals with process heads up to 1400 ft. (427 m.), 600 psi (40 bar), TDH at 3500 rpm, NPSH from 3 to 14 ft. (0.91 to 4.2 m.), and temperatures to 450° F. (232° C.). Another example of a regenerative turbine pump for use with the present apparatus is Dynaflow Regeneration Turbine Pump made by Dynaflow Engineering, Middlesex, N.J. Another example of a regenerative turbine pump for use with the present apparatus is Model MT5003P3T6 made by Warrender, LTD., Wood Dale, Ill.

Continuing downstream of the pump casing and referring to FIGS. 1A and 1B, the pumped process water, now containing bubbles of and dissolved carbon monoxide, methane and hydrogen, passes out of the pump casing and through the pump discharge 68. The pump discharge 68 also preferably has a smooth, straight-walled bore with minimal change in internal diameter across the pumped media flow path and also preferably intersects the casing tangentially, so that turbulence is minimized.

Next the fuel gas laden process water passes through the optional discharge bubble detection apparatus 71. Discharge bubble detection apparatus 71 may include sensor element 71a, transmitter 71b and indicator 71c. As with inlet detection, the controller 100 can monitor conditions or properties of the process water in order to determine the correct speed setpoint 109 of the regenerative turbine pump 60 and the correct pressure setpoints 108 of discharge pressure control valve 80. To control bubble collapse rate and prevent incomplete reaction, detection and measurement of the number and size of carbon dioxide bubbles that remain in the process water stream discharged from the regenerative turbine pump 60 can be used to recalculate a correction to the pump speed or the discharge pressure setpoints 109, 108. It is expected that the rate of bubble collapse within the regenerative turbine pump 60 will increase as the maximum pump discharge pressure setpoint 108, as controlled by the regenerative turbine pump 60 impeller (FIG. 2C, 63) speed and regulated by the discharge pressure control valve 80, is increased.

Once bubble generation is underway, the optional discharge bubble detection apparatus 71 is used to detect and measure any bubbles that remain in the process water flow downstream of the regenerative turbine pump 60. If bubbles are detected in the discharge flow, where none should be present, or if bubbles larger then those that should be present are detected, then the discharge pressure setpoint can be increased.

An increase in regenerative turbine pump 60 discharge pressure can be accomplished in at least two ways. First, where the current discharge pressure setpoint, as regulated by the discharge pressure control valve 80, is less than the shutoff, or maximum, pressure of the regenerative turbine pump 60 while operating at its current speed setpoint, then the discharge pressure control valve 80 is used to increase the discharge pressure setpoint. Second, where the current discharge pressure setpoint is equal to the maximum possible at the current regenerative turbine pump speed setpoint, then the pump impeller 63 speed setpoint is increased. Consequently, the maximum possible discharge pressure setpoint is increased. Once the regenerative turbine pump impeller 63 speed setpoint is increased, additional upward discharge pressure increase and regulation is accomplished by increasing the discharge pressure setpoint of the discharge pressure control valve 80. In this way, the discharge pressure setpoint and the regenerative turbine pump 60 speed setpoint can be manipulated independently, allowing a particular application to achieve and sustain a particular discharge pressure setpoint, as required to collapse the generated bubbles, while at the same time varying the regenerative turbine pump impeller 63 speed setpoint. This enables the precise timing of the impeller buckets (FIG. 2A, 64) passing by the regenerative turbine pump 60 inlet to the number of bubbles output by the bubble generation apparatus 30, enabling the aforementioned desired collapse of bubbles singularly in an isolated way at, for example, their eigenfrequencies.

Again, continuing with FIGS. 1A and 1B, the process water now passes by the discharge pressure 113 and temperature sensors 114, as well as any other detectors or sensors that are installed to measure the composition or condition of the process water in the discharge pipe 75. The discharge pressure and temperature sensors 113, 114 may include sensor elements 113a, 114a, transmitters 113b, 114b, and indicators 113c, 114c. The process water flows through the discharge pressure control valve 80 and into the gas liquid separator 90. The discharge pressure control valve 80 is configured for regulation of process water pressure upstream of the discharge pressure control valve 80, that is, pressure regulation occurs in the discharge pipe 75 between the regenerative turbine pump 60 and the discharge pressure control valve 80. The inlet pressure control valve 24, on the other hand, regulates pump inlet pipe 22 pressure downstream of the inlet pressure control valve 24. In this way, the pressure to collapse the carbon dioxide bubbles is set and regulated by combined manipulation of the discharge pressure control valve 80 pressure setpoint 108 and the regenerative turbine pump 60 speed setpoint 109.

Finally, the process water containing dissolved fuel gas and bubbles of fuel gas passes into the gas liquid separator 90. Fuel gas is drawn out of the separator by action of the fuel gas transfer pump 92. De-gasification is controlled by maintaining a stable light vacuum (for example, ˜0.95 atm) within the separator. Fuel gas transfer pump control PLC 206 receives the separator 90 pressure data from the gas liquid separator pressure sensor 209, which may include pressure element 209a, pressure transmitter 209b, and pressure indicator 209c and uses this value as a process variable, continuously recalculating a speed setpoint 217 for the fuel gas transfer pump 92. The pressure process control variable is transmitted to the fuel gas transfer pump speed control PID 207 along with the currently calculated fuel gas transfer pump speed setpoint 217. PID 207 calculates and transmits an analog or digital signal representing the desired fuel gas transfer pump 92 speed setpoint 217 to the fuel gas transfer pump speed controller 208 which varies the delivered NC power frequency such that the fuel gas transfer pump 92 rotates at the speed setpoint 217. PLC 206 communicates directly with PLC 211, providing fuel gas production rate data to PLC 211 for use in overall process efficiency calculation and setpoint adjustment, and for process interruption and/or operational parameter adjustments in response to inefficient or improper operation as determined by conditions in the gas liquid separator 90. The gas liquid separator liquid level controller 94 operates the process water transfer pump 96 as required, returning process water through the process water return pipe 98 to the process water supply 20. The fuel gas transfer pump 92 delivers the carbon monoxide, methane and hydrogen fuel gas to either a fuel gas storage vessel (not shown) or to a fuel consuming device or process.

The process water and fuel gas handling subsystems of the apparatus may be outfitted with relief, bypass or other unloader valves (not shown) and other safety devices and features as required by the nature of the particular application. Additionally, inlet and discharge isolation (not shown) and check valves (not shown) should be installed where required to prevent improper flow and to provide for apparatus isolation, testing and service.

The controller 100 provides both operation condition detection and control services for the apparatus. In its role as a pump motor control system, the controller 100 generates and transmits to the variable frequency drive 126 motor controller start, stop and other variable frequency drive function commands and the pump motor speed setpoint 109 signal. The variable frequency drive 126 is connected to and provides power for the regenerative turbine pump 60 motor, controlling the motor's rotational speed and consequently the coupled regenerative turbine pump 60 speed as directed by the motor speed control pilot signal received from the controller 100. In addition to regenerative turbine pump 60 motor control, the controller 100 operates the motorized inlet valve pilot regulator 26 and motorized discharge valve pilot regulator 82, varying the inlet pressure, as regulated by the inlet pressure control valve 24, and the discharge pressure, as regulated by the discharge pressure control valve 80. The controller 100 also monitors and can record apparatus subsystem parameter values received from the pump inlet pipe 22 and discharge pipe 75 sensors 40, 71, 111, 112, 113, 114 and recalculates setpoints related to the system's operation as required by a particular application.

The controller logic 101 stored and executed by the controller logic PLC 211 provides the controller 100 and the apparatus operational sequence and other functions. The controller logic 101 can be changed as required to include functions specific to a particular apparatus configuration. FIGS. 1A and 1B show an exemplary set of detectors and controlling devices for implementing the operational methods explained herein. Other configurations of the apparatus are possible using other equipment, detectors and controllers not shown, or not using some of the shown apparatus subsystems or components. To accommodate possible physical configuration changes to the apparatus, the controller logic 101—those programming instruction pertaining to the installed apparatus and controller 100 device identification, state detection, control and task distribution—can be altered such as by uploading the program to an external computer or device for storage, required modification and subsequent download back to controller logic PLC 211. Alternately, separate instances of apparatus configuration specific controller logic 101 can be stored locally in the controller 100 or in an external computer or device, to be uploaded or executed as required by the controller logic PLC 211. Additionally, it may be necessary to change the operational sequence of the apparatus for a particular application. Carbon dioxide reduction logic 106—such as the order of sensor or detector evaluation, the order of setpoint modification, the algorithms for setpoint modification, or algorithms used to identify and recover from operational fault states—may be stored in or accessible to and executed on the controller logic PLC 211. As with controller logic 101, a unique version of the carbon dioxide reduction logic 106 can be stored locally in the controller or on an external device or carbon dioxide reduction logic 106 can be uploaded to an external device or computer, modified, and downloaded back to the controller logic PLC 211. Inlet pressure setpoint 107 and discharge pressure setpoint 108 data that describe operational parameters such as pressure, temperature and bubble properties, and pump motor speed setpoint 109 data, are provided as individual values, independent or dependant values or value ranges or algorithms used to calculate values or value ranges, may be stored with or accessible to controller logic PLC 211 and can be uploaded and downloaded to an external device or computer. In each case, where data or program code stored in or accessible to the controller logic PLC 211 is to be modified, rather than uploading, modifying and downloading existing setpoint data 107, 108, 109 controller logic 101 or carbon dioxide reduction logic 106 program code, it is also possible to access and modify this information on or accessible to the controller 100 directly using an external device or computer. In addition, an operator control panel (not shown) can be provided to allow manual control of the apparatus, manual entry of setpoint data 107, 108, 109, manual manipulation of or interaction with controller logic 101 or carbon dioxide reduction logic 106, or manual control of invention subsystems or components directly, such as the pressure control valves 24, 80 or the variable frequency drive 126.

The external link PLC 118 provides a direct connection and controller 100 interface to an external device or computer, direct access to the data and program code stored on or with the controller PLC 211 from an external device, and logic for automated or externally directed upload and download of carbon dioxide reduction logic 106 and controller logic 101 and setpoint data 107, 108, 109. Where the apparatus is part of a larger system, the controller logic 101 can incorporate steps to accept directives from and report operational parameter values and status to an external system, computer or device. In these cases, the external link PLC 118 can be configured and programmed to marshal this external communication and control between the external device or computer and the controller logic PLC 211.

Note that although FIGS. 1A and 1B depict discrete PLC's in the controller 100, such as one for external link 118 and one for controller logic 211, as well as others such as inlet pressure control PLC 200, discharge pressure control PLC 102, pump motor speed control PLC 104, these functions could be combined in a single PLC, a personal computer (PC) such as PC 119 or other similar device. In addition, while the external link PLC 118 and controller logic PLC 211, as well as the other PLC's 200, 102, 104 and PID's including inlet pressure control PID 201, discharge pressure control PID 103 and pump motor speed control PID 105, are shown in FIGS. 1A and 1B to be incorporated into a single controller, devices performing these functions could be installed in separate locations as part of separate controllers—this alternate control component arrangement may be likely where the apparatus is incorporated into a larger overall process or system. Also, while controller logic 101 and carbon dioxide reduction logic 106 are depicted in FIGS. 1A and 1B as residing in and executing on the controller logic PLC 211, it is possible that the controller logic 101 could reside in and execute on a different PLC, PC or other similar device than the one that stores and runs carbon dioxide reduction logic 106, and these separate PLC or alternate devices could also reside in separate controllers. Similar variation in component function distribution, grouping or placement is possible with the other sensor-transmitter-indicator devices such as 40, 71, 110, 111, 112, 113, 114, 135, PLC 200, 102, 104 and PID 201, 103, 105 as well. The controller 100 component arrangements and functions rendered in FIGS. 1A and 1B are exemplary of stand-alone, self-contained operation and control of the apparatus, for use as depicted when the system is configured and connected upstream and downstream as shown, or as a design feature guide for different physical configurations of the invention or where the invention is incorporated as a single element or step in a multi-function or multi-step process. Consequently, in the discussion of the controller 100 component functions contained herein, it should be understood that where a particular PLC, PID or other device with specific functions is discussed, a PC, PLC or other functionally equivalent device could be substituted for the one described. Additionally, discrete functions performed by the described controller 100 component may be performed by another device along with other unrelated functions.

Individual subsystem controls and instrumentation may be grouped in the controller 100 by related function and may be monitored and controlled as a group by a discrete individual PLC, PC or other similar device. This permits discrete subsystem data storage and programming. In this way the addition, configuration change or removal of subsystems or subsystem components requires only the addition, change, reprogramming or deletion of those corresponding elements of the controller 100 directly responsible for the state detection or control of the affected subsystem or component.

Controller logic PLC 211 executes the controller logic 101 and carbon dioxide reduction logic 106 program instructions that direct the operational sequence of the apparatus subsystems, as previously described, and responds to external computer or operator requests. PLC 211 also handles operational sequence interruptions and operational parameter value data requests generated by the subsystem controllers PLC 200, PLC 102 and PLC 104. PLC 211 may also be used for handling and maintaining overall operational status information and requests for this information transmitted from the subsystem controllers and for relaying setpoint and subsystem status information between the apparatus subsystems as they request such data or status for their operation. Operational limit conditions, error states and other events than occur during invention operation that require the apparatus to change operational mode, halt, reset or communicate an operational or component status or alarm to an operator, external computer or device may also be handled by the controller logic PLC 211.

The inlet pressure control PLC 200 receives from controller logic PLC 211, initially and periodically as required, inlet pressure setpoint 107 data, as values, value ranges or as an algorithm used to calculate a setpoint value or value range using pump inlet pipe 22 sensor 40, 111, 112 data. The inlet pressure setpoint 107 data values specify, for a particular application, the required pressure and temperature of the pumped media or the required number and size of bubble that emit from the bubble generation apparatus 30, or both. Additionally, inlet pressure setpoint 107 data may be provided for other properties of the pumped media at the pump inlet pipe 22 or of the generated bubbles, if apparatus to measure these are present. Inlet pressure control PLC 200 receives ranged analog or digital signal input from sensor transmitters whose elements are mounted at the bubble generation apparatus 30 inlet, such as the inlet pressure transmitter 111b and the inlet temperature transmitter 112b. Inlet pressure control PLC 200 is also connected to and receives a ranged analog or digital signal input from the inlet bubble detection apparatus 40, which may be interpolated to represent the size and number of bubbles detected. Utilizing inlet pressure control, carbon dioxide reduction logic 106 and inlet pressure setpoint 107 data, inlet pressure control PLC 200 monitors pump inlet pipe 22 temperature, pressure, and other properties, as well as bubble number and size and recalculates continuously during operation the required target inlet pressure setpoint 107. During operation, the inlet pressure setpoint 107 required to maintain uniform, stable, continuous operation of the bubble generation apparatus 30, as specified by a particular application, may vary due to pump inlet pipe 22 or discharge pipe 75 turbulence, pumped media flow rate or temperature change, pump speed change, discharge pressure change, or change in another property of the bubbles or pumped media. As these changes occur, the properties of the generated bubbles may vary outside an application's specified range. Inlet pressure control PLC 200 can calculate a new inlet pressure setpoint 107 expected to mitigate the bubble property changes and restore bubble production to the application's specifications. This processing continues until interrupted by controller logic PLC 211, which can provide new inlet pressure setpoint 107 data or direct inlet pressure control PLC 200 to set a specific inlet pressure setpoint 107 and stop processing. In addition, the controller 100 can provide panel mounted operators (not shown) and pump inlet pipe 22 sensor indicators—inlet pressure indicator PI 111c, inlet temperature indicator TI 112c—that enable manual control of the inlet pressure setpoint 107. An inlet pressure setpoint 107 manually input via a panel operator may be processed by controller logic PLC 211 as setpoint data from an external device or computer would be and as a specific inlet pressure setpoint 107 with no additional processing by inlet pressure control PLC 200.

Once an inlet pressure setpoint 107 is calculated by inlet pressure control PLC 200 it is transmitted together with the current inlet pressure process variable to inlet pressure control PID 201. PID 201 continuously receives updated inlet pressure process variable and setpoint pressure values. PID 201 then calculates and transmits a ranged analog or digital signal corresponding to the position of the motorized inlet valve pilot regulator 26 required to set the inlet pressure control valve 24 to the inlet pressure setpoint 107. If the motorized inlet valve pilot regulator 26 is equipped with its own controller, inlet pressure control PID 201 will transmit a ranged analog or digital signal representing the inlet pressure setpoint 107 to the motorized inlet pilot valve's controller, which will in turn calculate the required pilot valve position to set the inlet pressure control valve 24 to the inlet pressure setpoint 107.

Alternately, controller logic PLC 211 can direct—either as part of its intrinsic logic or as commanded by an external computer or device or operator—inlet pressure control PLC 200 to use the analog or digital signal from the inlet bubble detection apparatus 40 as the inlet pressure control PID 201 process variable. Two sequential operational modes are employed to implement this control technique. First, inlet pressure control PLC 200, using the aforementioned inlet pressure setpoint 107 handling techniques and in conjunction with inlet pressure control PID 201, sets the inlet pressure so that the bubble generation apparatus 30 is operating within application specifications for bubble number and size. Second, the interpolated analog or digital signal from the inlet bubble detection apparatus 40 corresponding to the optimal bubble properties is captured and used as the controlling setpoint in place of the inlet pressure setpoint 107. This captured setpoint signal is continuously transmitted together with the signal from the inlet bubble detection apparatus 40, which in this case is used as the process variable, to inlet pressure control PID 201. PID 201 subsequently varies the position signal or pressure value transmitted to the motorized inlet valve pilot regulator 26, and consequently the regulated pressure at the bubble generation apparatus 30 inlet, in response to changes in bubble properties. In this way closed loop control of the inlet pressure required by the bubble generation apparatus 30 is continued in response to the inlet bubble detection apparatus 40 signal. Inlet pressure control PLC 200 can continue its control operation in this alternate mode or switch back to inlet pressure setpoint 107, and detected inlet pressure process variable based control of inlet pressure control PID 201.

Once the motorized inlet valve pilot regulator 26 position is set, the inlet pressure control valve 24 will maintain the set pressure without further pilot control adjustment. Consequently, in applications where repeated or continuous change in or adjustment of the inlet pressure setpoint 107 does not occur during normal operation, the inlet pressure control PID 201 can be omitted or replaced with a proportional-integral controller (PI) or other similar, simpler, proportional controller. It is understood, however, that in many applications, fine control of inlet pressure setpoint 107 variations will be required to sustain optimal apparatus operational parameter values.

Once bubbles are generated, they pass through the regenerative turbine pump 60 and are collapsed. Discharge pressure control PLC 102 and pump motor speed control PLC 104 both contribute to the process of pump discharge 68 and discharge pipe 75 pressure control. Discharge pressure control PLC 102 receives discharge pressure setpoint 108 data from controller logic PLC 211, initially and periodically, similar to inlet pressure control PLC 200, as values, ranges or algorithms to calculate the discharge pressure setpoint. Discharge pressure control PLC 102 receives and monitors ranged analog or digital signals from the discharge pipe 75 mounted sensors: discharge pressure sensor 113, discharge temperature sensor 114, and discharge bubble detection apparatus 71, as well as any other properties of the discharged process water that the apparatus might be additionally equipped to detect. Discharge pressure control PLC 102 continuously monitors the various aforementioned discharge pipe 75 process variables and recalculates the discharge pressure setpoint 108 required to collapse the bubbles at the rate and to the size required by the particular application. Similarly to bubble generation control, bubble collapse rate, final bubble size, or total bubble collapse control may require variable discharge pressure and pump rotation speed setpoints as operational parameters change. Discharge pressure control PLC 102 monitors discharge pipe process variables and continuously recalculates the discharge pressure setpoint 108 expected to mitigate any variance from bubble collapse specifications for an application.

Operation of the discharge pressure control valve 80, through operation of the motorized discharge valve pilot regulator 82 by discharge pressure control PID 103, as directed by pressure control PLC 102, utilize signal types, signal processing, process variable selection—such as discharge pressure or discharge bubble detection apparatus 71 signal—and operational considerations and control techniques similar to the analogous pump inlet pipe 22 pressure control accomplished by inlet pressure control PLC 200 and PID 201, motorized inlet valve pilot regulator 26 and inlet pressure control valve 24. Remote control of the discharge pressure setpoint 108 by an external computer or device can be relayed through controller logic PLC 211 and discharge pressure control PLC 102. Also, as is the case with manual pump inlet pipe 22 pressure control, manual discharge pipe 75 pressure control is possible using panel mounted operators (not shown) and the feedback from the panel mounted indicators including valve position indicator 110, speed indicator 134, valve position indicator 132, pump speed indicator 115, pump speed indicator 215, discharge pressure indicator 113, discharge temperature indicator 114, and discharge bubble detection apparatus indicator 71, and the motorized discharge valve pilot regulator 82 position indicator 116.

As hereinbefore set forth, the rotational speed setpoint 109 of the regenerative turbine pump 60 can be calculated by the controller 100 considering various factors. For example, the pump motor speed setpoint 109 must be high enough that the regenerative turbine pump 60, when its impeller (FIG. 2A, 63) is driven at the pump motor speed setpoint 109, will output a minimum pressure equal to or greater then the calculated discharge pressure setpoint 108. Subsequent to reaching the aforementioned minimum rotational speed, the pump motor speed setpoint 109 can be upwardly (or to a limited degree downwardly) adjusted so that the rate of the passage of the regenerative turbine pump 60 impeller's buckets (FIG. 2A, 64) matches the generated bubble production rate. In this way the bubbles flow singularly into the regenerative turbine pump's 60 impeller buckets.

To accomplish this, the controller 100 permits direct interaction between discharge pressure control PLC 102 and pump motor speed control PLC 104 and relays inlet bubble detection apparatus 40 interpolated data that provides the number of bubbles generated per minute (or other time interval) from inlet pressure control PLC 200, via the controller logic PLC 211, to pump motor speed control PLC 104.

Controller logic PLC 211, in addition to and in support of controller logic 101, also stores and distributes to the controller's subsystems PLC 200, 102, 104 upon request parametric data describing the operational performance characteristics and limits of the current configurations of the apparatus subsystems, including information about the installed regenerative turbine pump 60. This pump configuration information can include performance curve data based on the regenerative turbine pump's 60 rotational speed, providing specifications such as maximum discharge pressure, maximum rotational speed, or flow rate, horsepower requirement or NPSHr as a function of the rotational speed. This configuration data is used by the apparatus subsystems' control PLC 200, 102, 104 to verify setpoint data ranges and identify out of limit operational conditions and for operational error control. In addition to this standard pump performance curve information, data regarding the regenerative turbine pump 60 impeller's (FIG. 2A, 63) design is stored accessible to the controller logic PLC 211 and reported to pump motor speed control PLC 104, including the number of impeller buckets (FIG. 2A, 64) along the circumference of the pump's impeller.

Controller logic PLC 211, using the stored subsystem configuration data, controller logic 101, carbon dioxide reduction logic 106, and setpoint data 107, 108, 109, retrieves or calculates and then transmits at operation startup an initial discharge pressure setpoint 108 to the discharge pressure control PLC 102 and an initial pump motor speed setpoint 109 to the pump motor speed control PLC 104. The discharge pressure control PLC 102 then recalculates the discharge pressure setpoint 108 and transmits it, along with the process variable, either the pump discharge pipe 75 pressure sensor 113 signal or the discharge bubble detection apparatus 71 signal, to the discharge pressure control PID 103 so it can position the motorized discharge valve pilot regulator 82. If the bubble collapse rate is insufficient, or the final bubble size is greater than the invention application specification, or bubbles are to be totally collapsed and yet are still seen by the discharge bubble detection apparatus 71, then the discharge pressure setpoint 108 is increased. The newly calculated higher discharge pressure setpoint 108 is transmitted directly from discharge pressure control PLC 102 to pump motor speed control PLC 104. PLC 104 evaluates the current discharge pressure setpoint 108, and using the stored regenerative turbine pump performance data, in conjunction with the current pump motor speed setpoint 109, determines whether the maximum pressure at the current pump motor speed setpoint 109 is greater than the transmitted newly calculated higher discharge pressure setpoint 108. If the current pump motor speed setpoint 109 is too low, pump motor speed control PLC 104 calculates a new pump motor speed setpoint 109 to cause the regenerative turbine pump 60 to output at least the new discharge pressure setpoint 108 requested by the discharge pressure control PLC 102. Conversely, where the collapsed bubbles are too small or the calculated collapse rate is too great, the discharge pressure setpoint 108, and possibly the pump motor speed setpoint 109 can be lowered. Both of these processes can be implemented using a ranged analog or digital signal representing the speed setpoint, calculated by pump motor speed control PID 105 and transmitted to the variable frequency drive 126, which in turn varies the power frequency supplied to the pump's motor so that it rotates at the pump motor speed setpoint 109. The variable frequency drive 126 continuously returns operational status data, including current actual pump motor rotational speed, to the pump motor speed control PLC 104, which in turn relays this actual pump motor rotational speed data to the pump motor speed control PID 105 as the process variable. Once the final adjustment of the pump motor speed setpoint 109 is complete and bubble collapse occurs as desired, pump motor speed control PLC 104 can switch the process variable used by the pump motor speed control PID 105 from the actual rotational speed signal relayed from the variable frequency drive 126 to the bubble properties signal transmitted from the discharge bubble detection apparatus 71. As with inlet pressure control, the discharge pressure control PID 103 and the pump motor speed control PID 105 process variables can be switched as required between the actual discharge pipe pressure as detected by the discharge pressure sensor 113 or the discharge bubble detection apparatus 71 signal.

Once the minimum discharge pressure setpoint 108 for a bubble collapse rate and final bubble size for a particular application is achieved, fine pump motor speed setpoint 109 adjustment can commence. To accomplish this, pump motor speed control PLC 104 requests the current bubble generation rate from PLC 211, which in turn retrieves the current inlet bubble detection apparatus 40 bubble property values from inlet pressure control PLC 200. Once the current bubble production rate is retrieved, pump motor speed control PLC 104 then calculates the current impeller bucket (FIG. 2A, 64) passage rate as impeller (FIG. 2A, 63) revolutions per minute (or other time interval) multiplied by the total number of impeller buckets 64 on the impeller's circumference, yielding the total number of impeller buckets passing by the pump inlet 61 per minute or other time interval. With this bubble generation rate data and impeller bucket passage rate data, pump motor speed control PLC 104 continuously recalculates a new pump motor speed setpoint 109 that synchronizes the impeller bucket passage rate to the bubble generation rate so that the bubbles are collapsed singly in the impeller buckets 64.

It should be understood that to accomplish the timing between generated bubble and impeller bucket 64, and as a pre-requisite step of application design, the number of bubbles generated and number of impeller buckets should be coordinated so that the impeller can be rotated at the minimum speed to collapse the bubbles as required by an application using a particular impeller design and bubble generation apparatus 30 configuration.

The apparatus can be operated in one of at least three separate purpose modes: as directed by an external device or computer, or as directed by algorithms executed by controller logic PLC 211 using controller 100 and residing controller logic 101, carbon dioxide reduction logic 106 and setpoint data 107, 108, 109, or manually using panel mounted controls and indicators. In each of these three modes, the operational parameter values and setpoints, or the algorithms used to calculate them, as well as the useful subsystem process variable identities, are known and are input as controller carbon dioxide reduction logic 106 and setpoint data 107, 108, 109 intended and expected to achieve a particular operational result.

In another mode, where the controller 100 is used as a tool to determine the optimal operational parameter values and the identities of those process variables required to produce a particular functional result. In this experimental or application development operational mode, the setpoint data 107, 108, 109 submitted represent test value ranges, or are algorithms used to calculate test value ranges, and include target performance specifications for bubble production and collapse. In this mode, the carbon dioxide reduction logic 106 can provide both an operational test sequence algorithm that controls how each setpoint should be varied across the submitted setpoint data 107, 108, 109 range, as well as an algorithm and criteria to evaluate each set of operational parameter values against the target application performance specifications. During test execution, carbon dioxide reduction logic 106 stores those operational setpoints that provide useful results, either a good fit or a poor match to the target performance.

The controller 100 can be used as an analytical tool to determine the optimal operational parameter values required for producing bubbles of a certain size and collapsing them at a specific rate to plasma hot spots or as required by a particular application of the invention. The controller 100 allows automatic sequential execution of operational trials using electronically stored inlet and discharge pressure setpoint values or value ranges and pump speed setpoint values or value ranges that may produce desirable performance characteristics, as required by a particular application. The controller 100 automatically recalculates and varies the operational setpoints using the originally input setpoint values or value ranges and value modification algorithms residing in the controller. Alternately, the operational trials could be directed using an external computer, PLC or other functionally equivalent device to submit test setpoint data 107, 108, 109 and test application logic to controller PLC 211, through external interface PLC 118. Once testing is complete, result data can be read by or uploaded to an external computer or device for storage or further analysis. Rather than storing only criteria matching operational test data, all result data could be stored, locally in the controller 100 or on a remote computer or storage device for further analysis. The controller 100 concurrently records and subsequently analyses the trial operational results. In this way, an application protocol describing the operational conditions and process variable selections most likely to produce a desired result with the apparatus can be developed using the apparatus and controller 100 as reporting those setpoint combinations yielding desirable or best fit operational characteristics using controller residing result evaluation algorithms.

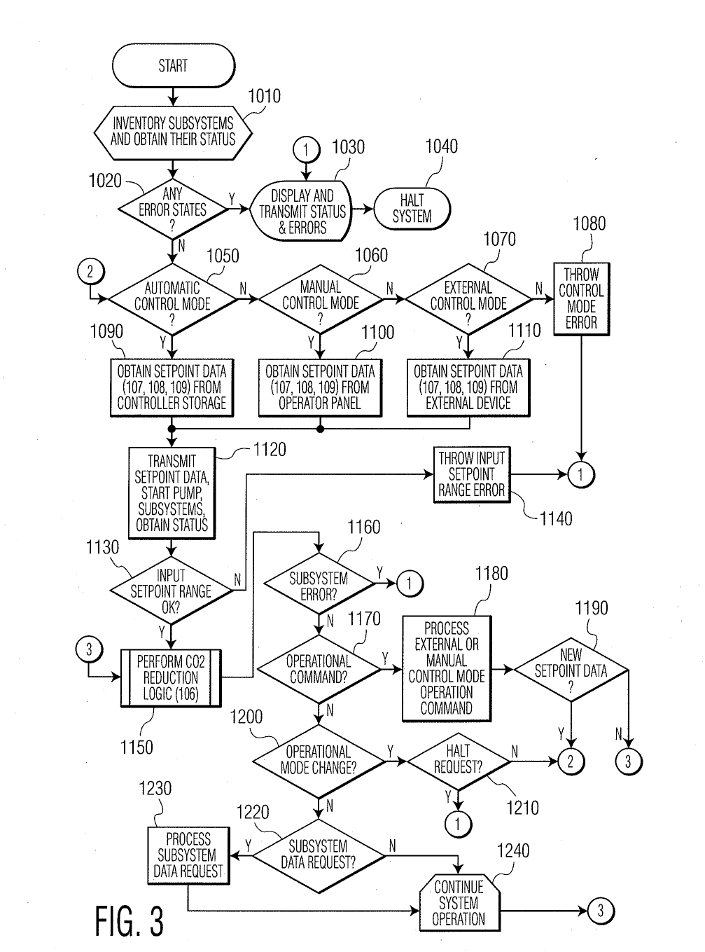

FIG. 3 is a flowchart showing processing steps that are carried out by the controller logic 101 of the present invention. Beginning in step 1010, the controller logic 101 inventories subsystems and obtains the status of the subsystems. A determination is made in step 1020 as to whether any errors are present. If errors are present, in step 1030 the errors are displayed and the status of the errors is transmitted for review, and then the system is halted in step 1040. Otherwise, if there are no errors in step 1020, a determination is made as whether to initiate automatic control mode in step 1050. If a negative determination is made, in step 1060 another determination is made as to whether to initiate manual control mode. If a negative determination is made, yet another determination is made as to whether to initiate external control mode in step 1070. If external control mode is not executed, a control mode error is transmitted (“thrown”) in step 1080 and the processing reverts to step 1030 in which error stats are displayed and the status is transmitted.

If automatic control mode is initiated in step 1050, the setpoint data 107, 108, 109 is obtained from controller in step 1090, and step 1120 occurs. If manual control mode is initiated in step 1060, the setpoint data 107, 108, 109 are obtained from an operator panel in step 1100, and step 1120 occurs. If external control mode is initiated in step 1070, the setpoint data 107, 108, 109 are obtained from an external device in step 1110, and step 1120 occurs. In step 1120, the setpoint data is transmitted, the pump 60 and subsystems are started and the status is obtained. Thereafter, in step 1130 a determination is made as to whether the setpoint range is acceptable. If a negative determination is made, an input setpoint range error is thrown in step 1140, and the processing reverts to step 1030. If the setpoint range is acceptable, carbon dioxide reduction logic 106 is performed in step 1150.

Next, in step 1160, a determination is made as to whether a subsystem error exists. If a positive determination is made, the processing reverts to step 1030. If no errors exist, a determination is made as to whether to initiate operational command in step 1170. If a positive determination is made, external or manual control mode operation command is processed in step 1180, and then, in step 1190, a determination is made as to whether to initiate new setpoint data. If a positive determination is made, the processing reverts to step 1050. If not, the processing reverts to step 1150.

If operational command is not initiated in step 1170, a determination is made as to whether there is a change in operational mode in step 1200. If a positive determination is made in step 1200, a determination is then made as to whether to halt the request in step 1210. If a positive determination is made, the processing reverts to step 1030. Otherwise, the processing reverts to step 1050. If operational mode change is not performed in step 1200, a determination is made as to whether to request subsystem data in step 1220. If a positive determination is made, the subsystem data request is processed in step 1230, and the operation of the system continues in step 1240, and the processing reverts to step 1150. Otherwise, the operation of the system continues in step 1240, and the processing reverts to step 1150.

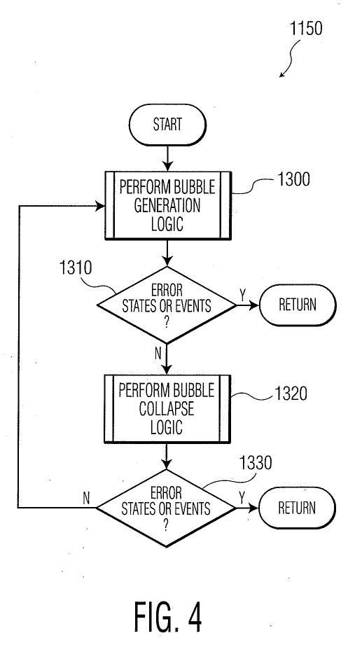

FIG. 4 is a flowchart showing processing steps of the carbon dioxide reduction logic 106. Beginning in step 1300, bubble generation logic is performed. In step 1310, a determination is made as to whether any error states or events are present. If any error states or events are present, the processing reverts to step 1150. Otherwise, bubble collapse logic is performed in step 1320. In step 1330, a determination is made as to whether any error states or events are present. If any error states or events are present, the processing reverts to step 1150. If step 1330 determines that there are no error states or events, control returns to step 1300.

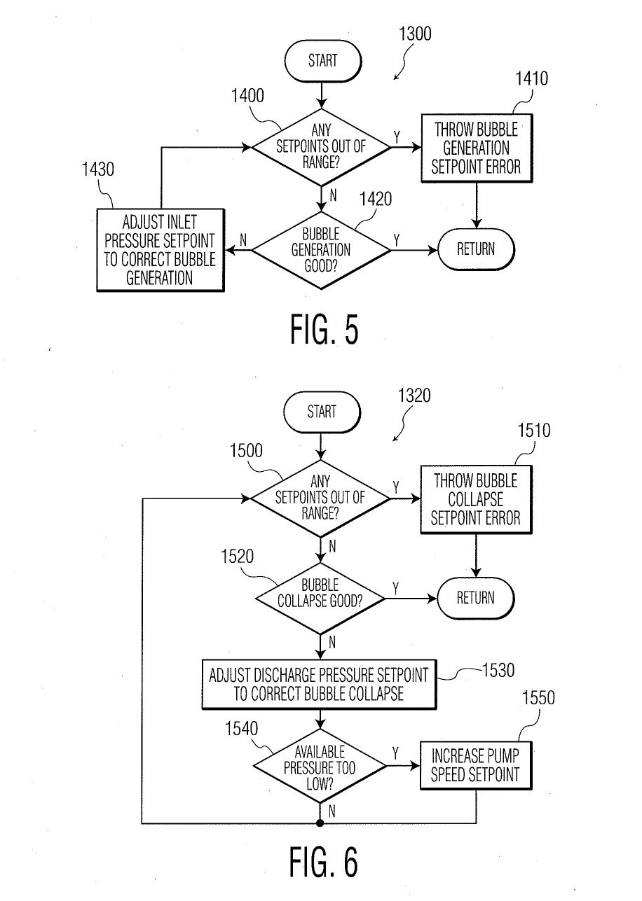

FIG. 5 is a flowchart showing processing steps of the bubble generation logic. Beginning in step 1400, a determination is made as to whether any setpoints are out of range. If a positive determination is made, a bubble generation setpoint error is thrown in step 1410 and the processing reverts to step 1300. Otherwise, a determination is made as to whether the bubble generation is acceptable in step 1420. If a positive determination is made, the processing reverts to step 1300. If the bubble generation is not acceptable, the inlet pressure setpoint is adjusted to correct bubble generation in step 1430, and the process reverts to step 1400.

FIG. 6 is a flowchart showing processing steps of the bubble collapse logic. Beginning in step 1500, a determination is made as to whether any setpoints are out of range. If a positive determination is made, a bubble collapse setpoint error is thrown in step 1510 and the processing reverts to step 1320. Otherwise, a determination is made as to whether the bubble collapse is acceptable in step 1520. If a positive determination is made, the processing reverts to step 1320. If the bubble collapse is not acceptable, the discharge pressure setpoint is adjusted to correct bubble collapse in step 1530. Next, in step 1540, a determination is made as to whether the available pressure is too low. If a negative determination, the process reverts to step 1500. Otherwise, the pump speed setpoint is increased in step 1550, and the process reverts to step 1500.

It will be understood that the embodiments described herein are merely exemplary and that a person skilled in the art may make many variations and modifications without departing from the spirit and scope of the invention. All such variations and modifications are intended to be included within the scope of the invention as defined by the appended claims.

What is claimed is:

1. An apparatus for producing fuel gas comprising:
an inlet pipe for delivery of a fluid;
a bubble generator for producing a linear stream of bubbles;
an injector for injecting the bubbles into the fluid; and
a regenerative turbine pump having an inlet for receiving fluid containing bubbles, and an impeller blade defining a plurality of buckets, the buckets receiving a single bubble which is collapsed to form a fuel gas.

2. The apparatus of claim 1, further comprising a gas liquid separator for separating the fuel gas from the fluid.

3. The apparatus of claim 2, further comprising a fuel gas transfer pump for moving the fuel gas from the gas liquid separator.

4. The apparatus of claim 1, wherein the means for moving the bubbles individually comprises a sensor sensing a process parameter associated with operation of the pump, and a controller in communication with the sensor, the controller processing the process parameter and adjusting operation of the pump based upon processing the process parameter.

5. The apparatus of claim 4, wherein the process parameter comprises at least one of the rotational speed of the impeller buckets, discharge pressure, and temperature.

6. The apparatus of claim 1, further comprising a pressure control valve in the inlet pipe for controlling flow.

7. The apparatus of claim 1, wherein the fluid is water.

8. The apparatus of claim 1, further comprising a bubble detection apparatus attached to the pump, the bubble detection apparatus detecting the number and size of the bubbles.

9. The apparatus of claim 1, wherein the bubble generator is an injector for injecting the bubbles into the fluid.

10. A method of creating methane fuel gas comprising:
producing carbon dioxide bubbles;
introducing the carbon dioxide bubbles into a stream of water;
delivering the stream of water with bubbles to a regenerative turbine pump having an impeller defining a plurality of buckets;
collapsing the bubbles to create an ionized gas and ionized liquid mixture containing hydrogen, hydroxyl radicals and hydroxide;
reacting the ionized gas and ionized liquid with carbon dioxide to produce carbon monoxide; and
reacting the carbon monoxide to produce methane.

11. The method of claim 10, further comprising collapsing the bubbles in isolation using a pressure pulse.

12. The method of claim 11, wherein the bubbles are collapsed in individual bucket chambers of a regenerative turbine pump.

13. The method of claim 12, wherein the bubbles are entrained in a helical flow in the chambers of the pump before collapse.

14. The method of claim 13, further comprising the step of monitoring pressure or flow rate of the stream of water using at least one sensor and a controller in communication with the at least one sensor.

15. The method of claim 14, further comprising the step of determining whether the pressure or the flow rate is within an acceptable range using the controller.

16. The method of claim 15, further comprising the step of adjusting operation of the regenerative turbine pump in response to monitoring of the pressure or the flow rate.

17. The method of claim 10 further comprising reacting methane and residual carbon dioxide and carbon monoxide with hydrogen to produce alkane gasses.

18. A system for producing fuel gas comprising:
an inlet pipe for delivery of a fluid to the pump;
a carbon dioxide gas supply for supplying carbon dioxide gas;
a bubble generator for producing a linear stream of bubbles;
an injector for injecting the bubbles into the fluid; and
a regenerative turbine pump having a plurality of buckets for receiving and collapsing the bubbles.

19. The system of claim 18, wherein the regenerative turbine pump includes an impeller blade defining the plurality of buckets, each bucket receiving a single bubble.

20. The system of claim 19, further comprising a sensor sensing a process parameter associated with operation of the pump, and a controller in communication with the sensor, the controller processing the process parameter and adjusting operation of the pump based upon processing the process parameter.

21. The system of claim 20, wherein the process parameter comprises at least one of the rotational speed of the impeller buckets, discharge pressure, and temperature.

22. The system of claim 21, further comprising a second sensor sensing a process parameter associated with operation of the bubble generation apparatus, and the controller in communication with the second sensor, the controller processing the process parameter and adjusting operation of the bubble generation apparatus based upon processing the process parameter.

23. The system of claim 22, wherein the process parameter comprises at least one of the size of the bubbles and rate of bubble formation.

24. The system of claim 18, further comprising a sensor sensing a parameter associated with producing the bubbles, and a controller in communication with the sensor, the controller processing the parameter associated with producing the bubbles.

25. The system of claim 24, further comprising a second sensor sensing a parameter associated with collapsing the bubbles, and the controller in communication with the second sensor, the controller processing the parameter associated with collapsing the bubbles.

26. The system of claim 18, further comprising a sensor sensing at least one parameter, and a controller in communication with the sensor, the controller processing the at least one parameter to allow automatic, sequential execution of operational trials.

27. The system of claim 26, wherein the at least parameter comprises at least one of the inlet pressure setpoint valve, discharge pressure setpoint valve, and pump speed setpoint valve.

28. The system of claim 27, wherein the pump includes an operational setpoint, the controller including value modification algorithms, the controller recalculating and varying the operational setpoint based on the at least one parameter.
